(19) United States
(12) Patent Application Publication
(10) Pub. No.: US 2022/0151678 A1
(43) Pub. Date: May 19, 2022

(54) ROBOTIC SURGICAL SYSTEMS WITH ELECTRICAL SWITCH FOR INSTRUMENT ATTACHMENT

(71) Applicant: Covidien LP, Mansfield, MA (US)

(72) Inventor: Jaimeen Kapadia, Cambridge, MA (US)

(73) Assignee: Covidien LP, Mansfield, MA (US)

(21) Appl. No.: 17/439,501

(22) PCT Filed: Mar. 11, 2020
(86) PCT No.: PCT/US2020/022036

§ 371 Date: Sep. 15, 2021

Related U.S. Application Data
(60) Provisional application No. 62/823,036, filed on Mar. 25, 2019.

(51) Int. Cl. A61B 18/12; A61B 18/14

(52) U.S. Cl. CPC A61B 18/1233; A61B 2018/0094; A61B 18/1445

(57) ABSTRACT

A robotic surgical system includes an electrosurgical energy source, a sterile interface module, and a surgical instrument. The surgical instrument has a housing and an elongated shaft that extends distally from the housing to an end effector. The housing supports a switch. The energy source is coupled to the surgical instrument to transmit electrical energy from the electrosurgical energy source to the switch. The switch is positioned to enable the electrical energy to be transmitted to the end effector of the surgical instrument when the sterile interface module is engaged with the switch. The switch is positioned to prevent the electrical energy from being to the end effector when the sterile interface module is disengaged from the switch.

### FIELD OF THE INVENTION

The present invention relates to robotic surgical systems used in minimally invasive medical procedures because of their increased accuracy and expediency relative to handheld surgical instruments.

### BACKGROUND

Robotic surgical systems have been used in minimally invasive medical procedures and can include robotic arm assemblies. Some robotic arm assemblies include one or more robot arms to which surgical instruments can be coupled. Such surgical instruments include, for example, electrosurgical forceps, cutting instruments, staplers, graspers, electrocautery devices, or any other endoscopic or open surgical devices. Prior to or during use of the robotic surgical system, various surgical instruments can be selected and connected to the robot arms for selectively actuating end effectors of the connected surgical instruments. Some of these surgical instruments utilize electrical energy, for example, to effectuate electrocautery with the end effector. The challenges associated with safely delivering electrical energy to the end effectors of these surgical instruments can add to the cost, size, and energy output of the robotic surgical system.

### SUMMARY

In accordance with one aspect, the present disclosure is directed to robotic surgical system. The robotic surgical system includes an energy source, a sterile interface module, and a surgical instrument. The surgical instrument has a housing and an elongated shaft that extends distally from the housing to an end effector. The housing supports a switch. The energy source is coupled to the surgical instrument to transmit electrical energy from the energy source to the switch. The switch is positioned to enable the electrical energy to be transmitted to the end effector of the surgical instrument when the sterile interface module is engaged with the switch. The switch is positioned to prevent the electrical energy from being transmitted to the end effector when the sterile interface module is disengaged from the switch.

In some embodiments, the sterile interface module may include a nub and the housing may define a nub recess. The switch may extend into the nub recess and may be configured to engage the nub. The switch may include a plunger that extends into the nub recess and a button disposed within the housing. The plunger may be positioned to selectively engage the button. The button may be coupled to a printed circuit board. The printed circuit board may flex toward the button when the plunger engages the button.

In embodiments, the switch may include a spring that urges the plunger into the nub recess when the nub is not engaged with the plunger. The switch may include a footing engaged with the housing and a button housing coupled to the footing. The button housing may be movable relative to the footing as the plunger moves relative to the nub recess. The spring may be engaged with the footing. The footing may include a flange that limits movement of the button housing relative to the footing.

In various embodiments, the switch may be a dome switch.

According to another aspect of the present disclosure, a surgical instrument for selective connection to a sterile interface module of a robotic surgical system is provided. The surgical instrument includes a housing configured for coupling to an electrosurgical energy source, an elongated shaft that extends distally from the housing, an end effector supported on the elongated shaft, and a switch supported by the housing. The switch is positioned to enable the electrical energy from the electrosurgical energy source to be transmitted to the end effector when the housing is coupled to the sterile interface module. The switch is positioned to prevent the electrical energy from being transmitted from the electrosurgical energy source to the end effector when the housing is uncoupled from the sterile interface module.

In embodiments, the housing may define a nub recess that is configured to engage a nub of the sterile interface module.

Other aspects, features, and advantages will be apparent from the description, the drawings, and the claims that follow.

### BRIEF DESCRIPTION OF THE DRAWINGS

Embodiments of the present disclosure are described herein with reference to the accompanying drawings, wherein:

FIG. 1 is a perspective view of a robotic surgical system in accordance with the present disclosure;

FIG. 2 is a perspective view illustrating proximal end portion of a surgical instrument of the robotic surgical system of FIG. 1 shown connected to a sterile interface module of the robotic surgical system;

FIG. 3 is a perspective view illustrating the surgical instrument and the sterile interface module of FIG. 2 separated from one another;

FIG. 4 is an enlarged, perspective view of a proximal end portion of the surgical instrument of FIGS. 2 and 3, the proximal end portion of the surgical instrument shown partially in cross-section to illustrate an electrical assembly of the surgical instrument;

FIG. 5 is a perspective view of the electrical switch assembly of FIG. 4;

FIG. 6 is a perspective view, with parts separated, of the electrical switch assembly of FIG. 5;

FIG. 7 is an enlarged, perspective view of a portion of a switch of the electrical assembly of FIGS. 5 and 6;

FIG. 8 is an enlarged, cross-sectional view of the sterile interface module of FIGS. 2 and 3 as taken along section line 8-8 shown in FIG. 3; and

FIGS. 9 and 10 are progressive views illustrating the surgical instrument of FIGS. 2 and 3 connecting to the sterile interface module of FIGS. 2 and 3.

### DETAILED DESCRIPTION

Embodiments of the presently disclosed robotic surgical system are described in detail with reference to the drawings, in which like reference numerals designate identical or corresponding elements in each of the several views. As commonly known, the term “clinician” refers to a doctor, a nurse, or any other care provider and may include support personnel. Further, as is used in the art, the term “distal” refers to a position, a direction, and/or a structure, which is closer to the patient, and the term “proximal” refers to a position, a direction, and/or a structure, which is farther from to the patient. In addition, directional terms such as front, rear, upper, lower, top, bottom, and the like are used simply for convenience of description and are not intended to limit the disclosure attached hereto.

In the following description, well-known functions or constructions are not described in detail to avoid obscuring the present disclosure in unnecessary detail.

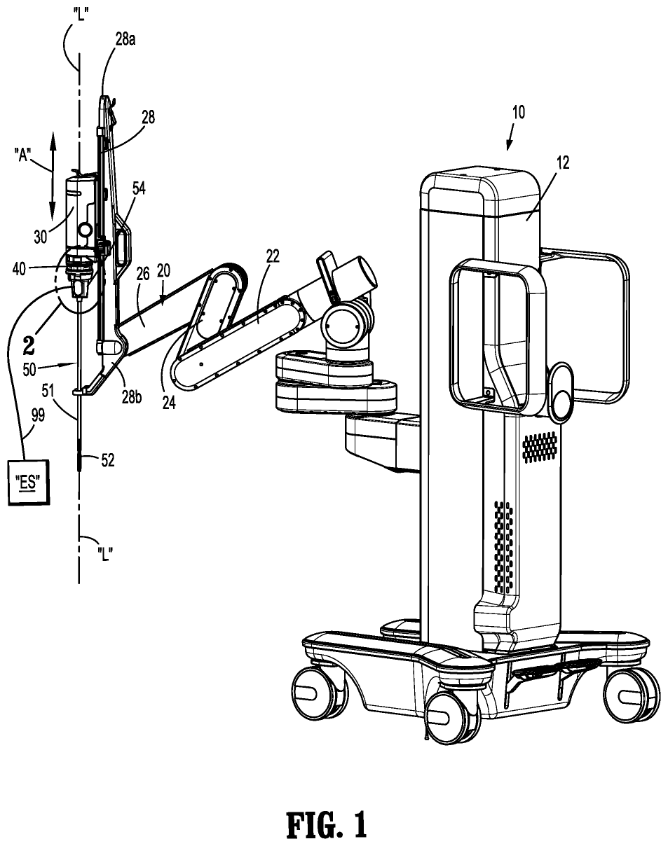

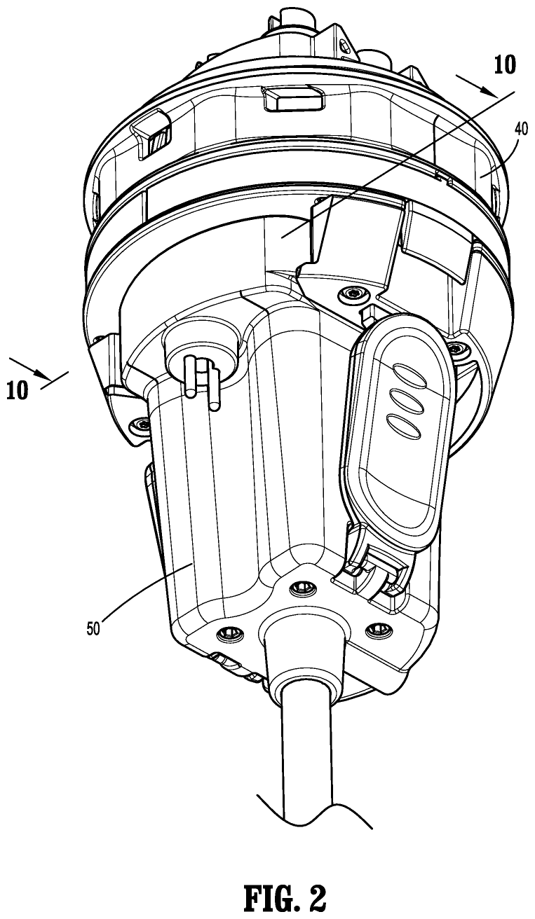

With brief reference to FIGS. 1 and 2, a robotic surgical system 10 includes a robotic arm assembly 20 that supports an instrument drive unit 30, a sterile interface module 40, and a surgical instrument 50 having an elongated shaft 51 with an end effector 52 (e.g., grasper, clip applier, stapler, vessel sealer, tack applier, etc.) supported on a distal end portion of elongated shaft 51 and housing assembly 54 supported on a proximal end portion of elongated shaft 51. For a more detailed description of similar surgical instruments, reference can be made to U.S. Patent Application Publication No. U.S. 2017/0224367 to Kapadia, and PCT Application Publication No. WO2017/205310 to Zemlok et al., the entire contents of each of which are incorporated by reference herein.

Robotic surgical system 10 employs various robotic elements to assist the clinician and allow remote operation (or partial remote operation) of surgical instrumentation such as surgical instrument 50. Various robotic arms, gears, cams, pulleys, electric and mechanical motors, etc. may be employed for this purpose and may be designed with robotic surgical system 10 to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

Robotic surgical system 10 includes a medical work station (not shown) that may be employed with one or more consoles positioned next to the operating theater or located in a remote location. In this instance, one team of clinicians may prep the patient for surgery and configure robotic surgical system 10 with surgical instrument 50 while another clinician (or group of clinicians) remotely controls surgical instrument 50 via the one or more consoles. As can be appreciated, a highly skilled clinician may perform multiple operations in multiple locations without leaving his/her remote console. This can be economically advantageous and a benefit to the patient or a series of patients. For a detailed description of exemplary medical work stations and/or components thereof, reference may be made to U.S. Pat. No. 8,828,023 and PCT Application Publication No. WO2016/025132, the entire contents of each of which are incorporated by reference herein.

With continued reference to FIG. 1, robotic arm assembly 20 of robotic surgical system 10 includes a cart 12 having robotic arms 22, 24, 26 that are pivotally coupled together and movable together and/or relative to one another and cart 12. Robotic arm 26 is coupled to a slide rail 28 that supports instrument drive unit (“IDU”) 30 and sterile interface module 40 for operating surgical instrument 50. IDU 30 defines a longitudinal axis “L” and is slidably supported on slide rail 28 and selectively axially movable along longitudinal axis “L,” as indicated by arrows “A,” between a proximal position adjacent a proximal end portion 28a of slide rail 28, and a distal position adjacent a distal end portion 28b of slide rail 28.

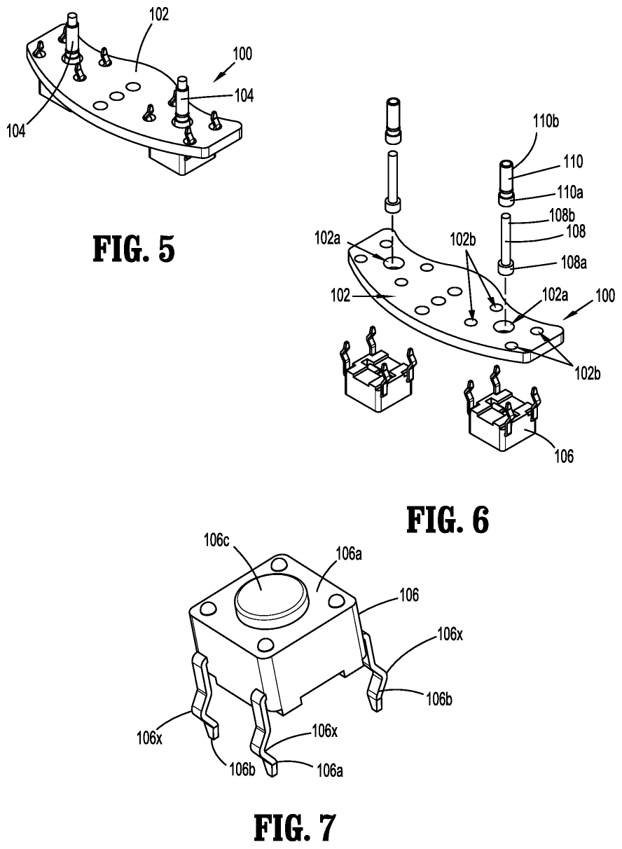

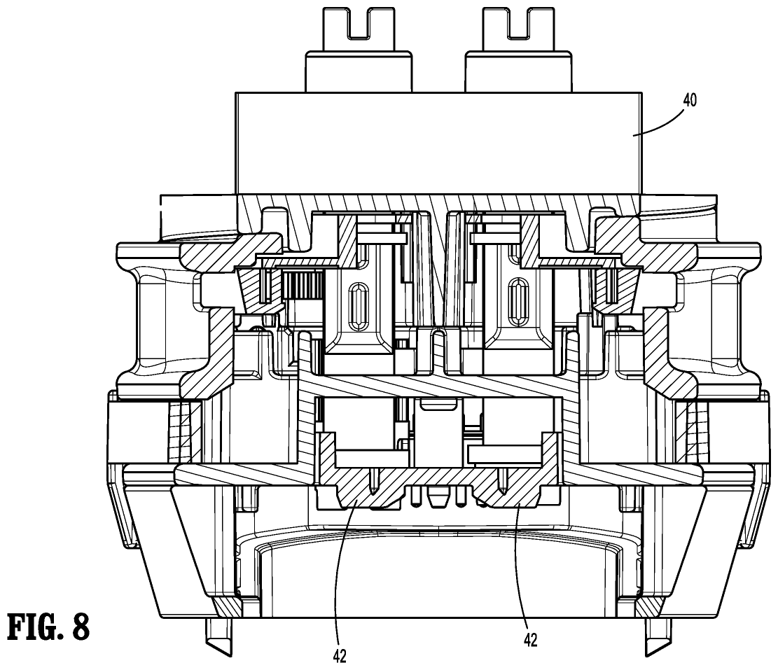

With reference to FIGS. 2-10, sterile interface module 40 of robotic surgical system 10 selectively interconnects IDU 30 and surgical instrument 50 to provide an interface between IDU 30 and surgical instrument 50. This interface advantageously maintains sterility, provides a means to transmit electrical communication between robotic surgical system 10 and surgical instrument 50, provides a means for transferring torque (e.g., rotational force) from robotic surgical system 10 (e.g., IDU 30) to surgical instrument 50 for performing a function (e.g., sealing, cutting, grasping, etc.) with surgical instrument 50 and/or provides a means to selectively attach/remove surgical instrument 50 to robotic surgical system 10 (e.g., for rapid instrument exchange).

In general, sterile interface module or (“SIM”) 40 supports a plurality of drive couplers 40a and electrical connectors 40b. SIM 40 further includes a semi-annular coupling cuff 40c that defines a U-shaped channel 40d for receiving surgical instrument 50 in a side-loading manner. SIM 40 also includes electrical nubs 42 that depend therefrom. For a more detailed description of similar sterile interface modules and components thereof, reference can be made to WO2017205308 by Zemlock et al., the entire contents of which are incorporated by reference herein.

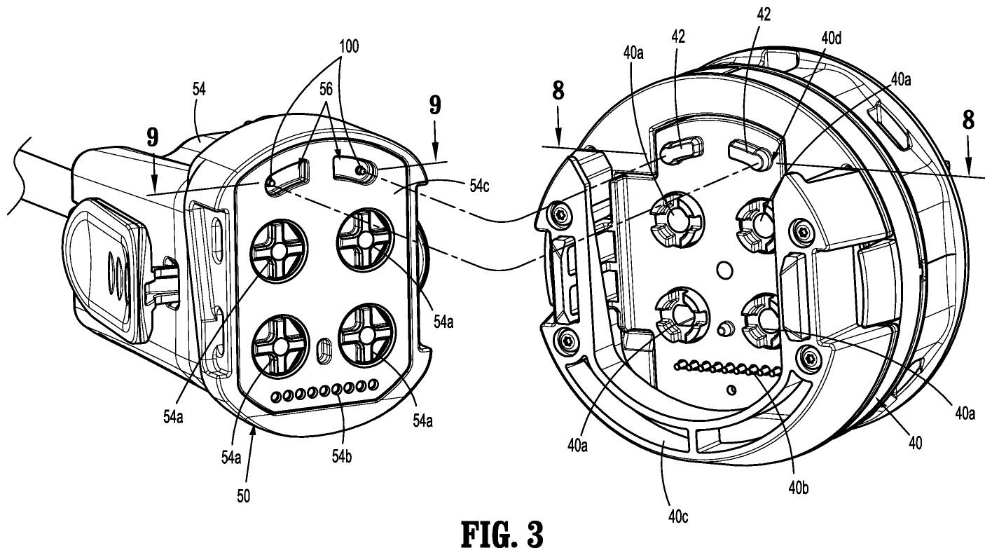

With reference to FIGS. 3, 4, and 9, housing 54 of surgical instrument 50 supports a plurality of driven couplers 54a that cooperate with drive couplers 40a of SIM 40 to operate end effector 52 (FIG. 1) of surgical instrument 50. Housing 54 also includes paddles 59 for selectively releasing housing 54 from SIM 40. Housing 54 further includes electrical connectors 54b that electrically communicate with electrical connectors 40b of SIM 40 to provide electrical communication between SIM 40 and surgical instrument 50. Housing 54 includes a cover 54c that defines nub recesses 56 in an upper surface of cover 54c. A lower surface of cover 54c includes projections 54d that extend into an upper chamber 54e of housing 54. Nub recesses 56 are configured to receive nubs 42 of SIM 40 therein. Housing 54 also includes an electrical switch assembly 100 supported within housing 54. Electrical switch assembly 100 extends into nub recesses 56 and is configured to engage nubs 42 of SIM 40 when nubs 42 are seated in nub recesses 56 of housing 54. Housing 54 includes an inner surface 55 with a shelf 55a for supporting electrical switch assembly 100 within upper chamber 54e of housing 54. Electrical switch assembly 100 is disposed in electrical communication with a connector assembly 58 by any number of wires or cables 58a of connector assembly 58. Connector assembly 58 is configured to electrically couple to an electrosurgical energy source “ES” (FIG. 1), such as an electrosurgical generator, to enable the electrosurgical energy source to deliver electrosurgical energy to electrical switch assembly 100. For a more detailed description of one example of an electrosurgical generator, reference can be made to U.S. Pat. No. 8,784,410, the entire contents of which are incorporated by reference herein.

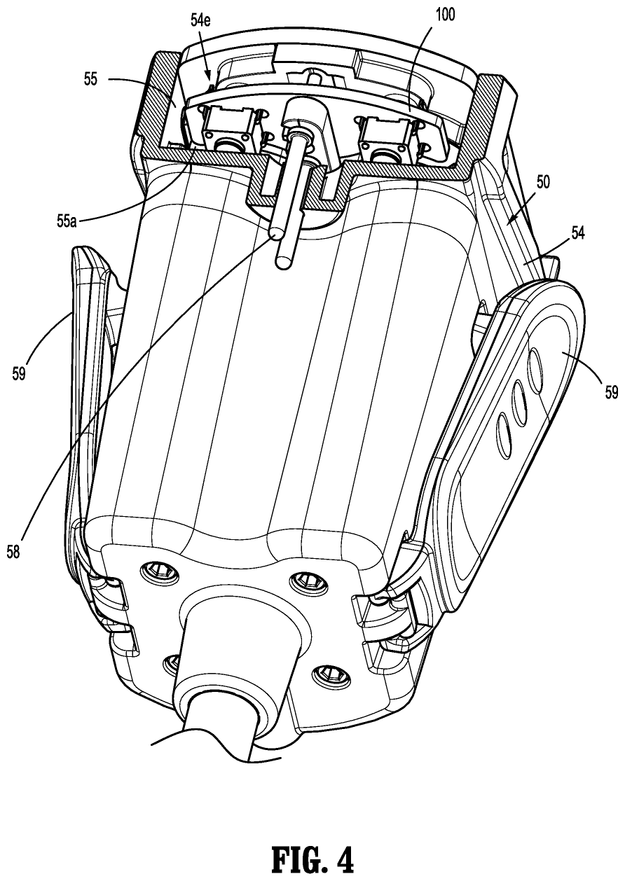

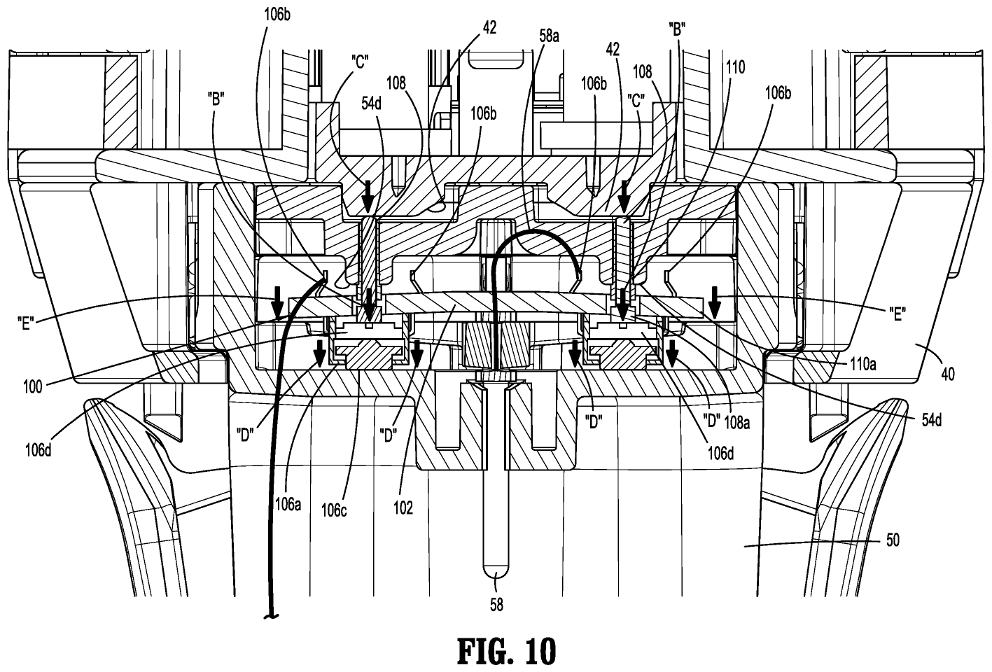

As seen in FIGS. 4-7, electrical switch assembly 100 of surgical instrument 50 includes a printed circuit board 102 and switches 104 (e.g., dome switches or the like) that are coupled to printed circuit board 102. Printed circuit board 102 defines plunger apertures 102a and button leg apertures 102b therethrough. Each switch 104 includes a button assembly 106, a plunger 108, and a sleeve 110. Sleeve 110 includes a base 110a and a tube 110b that extends from base 110a. Plunger 108 includes a base 108a and a rod 108b that extends from base 108a. Plunger 108 and sleeve 110 are secured to cover 54c and mounted within a plunger aperture 102a of printed circuit board 102 such that base 110a of sleeve 110 abuts projection 54d of cover 54c and base 108a of plunger 108 abuts button assembly 106. Rod 108b of plunger 108 is slidably movable through tube 110b of sleeve 110 so that plunger 108 is movable relative to sleeve 110, as indicated by arrows “B” (FIG. 10). In some embodiments, plunger 108 is coupled to sleeve 110 via a spring (e.g., a compression spring, not shown) disposed between plunger 108 and sleeve 110.

Button assembly 106 of electrical switch assembly 100 includes a button housing 106a and legs 106b secured to button housing 106a. Legs 106b extend from button housing 106a and are receivable within button leg apertures 102b of printed circuit board 102 to secure button housing 106a to printed circuit board 102. Legs 106b and/or button housing 106a can be secured to printed circuit board 102 using any known securement technique such as welding, soldering, crimping, etc. Each leg 106b includes an elbow 106x that engages a top surface of printed circuit board 102 to prevent legs 106b and button housing 106a from moving relative to printed circuit board 102. Button assembly 106 further includes a footing 106c supported in button housing 106a and engaged with shelf 55a of housing 54, a button 106d secured to button housing 106a and engaged with base 108a of plunger 108, and a spring 106e supported in button housing 106 between button 106d and footing 106c. Spring 106e is configured to spring bias button housing 106a toward plunger 108 for selectively extending rod 108b of plunger 108 into a nub recess 56 of cover 54c of housing 54. Footing 106c includes a nipple 106f that extends from footing 106c and engages spring 106e. Footing 106c further includes an annular flange 106g that extends radially outward from footing 106c and limits axial movement of button housing 106a relative to footing 106c.

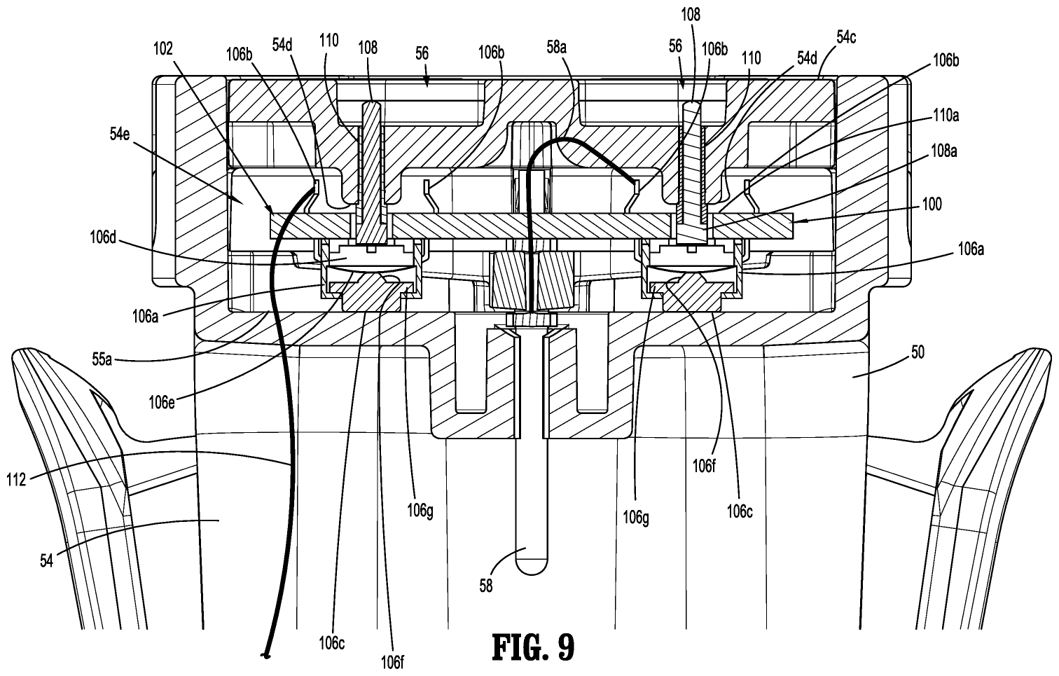

As seen in FIGS. 9 and 10, electrical switch assembly 100 is coupled to any number of electrical wires or cables 112 that connect to one or more legs 106 of button assembly 106. Wires 112 of electrical switch assembly 100 extend through surgical instrument 50 and are operatively coupled to end effector 52 (FIG. 2) of surgical instrument 50 to selectively deliver electrosurgical energy through surgical instrument 50 for effectuating an electrosurgical and/or an electrocautery procedure with end effector 52 of surgical instrument 50 when electrical switch assembly 100 is activated as illustrated in FIG. 10.

With reference to FIGS. 1-10, in use, housing 54 of surgical instrument 50 is side-loaded onto sterile interface module (SIM) 40 from an uncoupled position (FIG. 9) to a coupled position (FIG. 10). In the coupled position, drive couplers 40a of SIM 40 engage driven couplers 54a of surgical instrument 50 and electrical connectors 40b of SIM 40 engage electrical connectors 54b. As surgical instrument 50 couples to SIM 40, nubs 42 of SIM 40 slide into nub recess 56 of surgical instrument 50 so that nubs 42 depress plungers 108 of electrical switch assembly 100, as indicated by arrows “C.” As plungers 108 depress, plungers 108 move relative to sleeves 110 of electrical switch assembly 100 so that respective bases 108a of plungers 108 separate from bases 110a of sleeves 110. Depression of plungers 108 also causes bases 108a of plungers 108 to drive buttons 106d of button assemblies 106 toward footings 106c of button assemblies 106 to compress springs 106e of button assemblies 106 against nipples 106f of footings 106c of button assemblies 106 to thereby activate switches 104. Axial movement of buttons 106d of switches 104 causes button housings 106a of switches 104 to move axially relative to footings 106c of switches 104 toward shelf 55a of housing 54, as indicated by arrows “D.” Movement of button housings 106a of switches 104 toward shelf 55a of housing 54 causes printed circuit board 102 to flex toward shelf 55a of housing 54, as indicated by arrows “E.” In the coupled position, electrical switch assembly 100 is electrically activated such that when connector assembly 58 is coupled to energy source “ES,” electrical energy can be selectively transmitted through connector assembly 58 to electrical switch assembly 100 (by wire 58a), and through switch assembly 100 to end effector 52 of surgical instrument 50 (by wire 112).

Delivery of electrosurgical energy through surgical instrument 50 enables end effector 52 of surgical instrument 50 to effectuate an electrosurgical and/or an electrocautery procedure. For instance, surgical instrument 50 may be an electrosurgical forceps configured to deliver bipolar and/or monopolar energy through end effector 52 of surgical instrument 50. For a more detailed description of an exemplary electrosurgical forceps, reference can be made to U.S. Patent Application Publication No. 2017/0209206 by Kerr et al., the entire contents of which are incorporated by reference herein.

To deactivate electrical switch assembly 100, paddles 59 of housing 54 are actuated to separate housing 54 of surgical instrument 50 from SIM 40 via side unloading. As housing 54 of surgical instrument 50 is unloaded from SIM 40, nubs 42 of SIM 40 separate from nub recesses 56 of housing 54 so that springs 106e of button assemblies 106 of electrical switch assembly 100 cause plungers 108 of switches 104 to spring back from a depressed position (FIG. 10), where switches 104 are activated, to their original position (e.g., an undepressed position seen in FIG. 9), where switches 104 are deactivated—even if connector assembly 58 is still electrically coupled to energy source “ES” (FIG. 1) (e.g., where a cable 99 of energy source “ES” is plugged into connector assembly 58). Coupling and/or uncoupling of surgical instrument 50 to/from sterile interface module 40 can be repeated as desired.

As can be appreciated, securement of any of the components of the presently disclosed apparatus can be effectuated using known securement techniques such welding, crimping, gluing, fastening, etc.

Persons skilled in the art will understand that the structures and methods specifically described herein and illustrated in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, it is envisioned that the elements and features illustrated or described in connection with one exemplary embodiment may be combined with the elements and features of another without departing from the scope of the present disclosure, and that such modifications and variations are also intended to be included within the scope of the present disclosure. Indeed, any combination of any of the presently disclosed elements and features is within the scope of the present disclosure. Accordingly, the subject matter of the present disclosure is not to be limited by what has been particularly shown and described.

1. A robotic surgical system comprising:
an electrosurgical energy source;
a sterile interface module; and
a surgical instrument having a housing and an elongated shaft that extends distally from the housing to an end effector, the housing supporting a switch, the electrosurgical energy source coupled to the surgical instrument to transmit electrical energy from the electrosurgical energy source to the switch, the switch positioned to enable the electrical energy to be transmitted to the end effector of the surgical instrument when the sterile interface module is engaged with the switch, the switch positioned to prevent the electrical energy from being transmitted to the end effector when the sterile interface module is disengaged from the switch.

2. The robotic surgical system of claim 1, wherein the sterile interface module includes a nub and the housing defines a nub recess, and wherein the switch extends into the nub recess and is configured to engage the nub.

3. The robotic surgical system of claim 2, wherein the switch includes a plunger that extends into the nub recess and a button disposed within the housing, the plunger positioned to selectively engage the button.

4. The robotic surgical system of claim 3, wherein the button is coupled to a printed circuit board.

5. The robotic surgical system of claim 4, wherein the printed circuit board flexes toward the button when the plunger engages the button.

6. The robotic surgical system of claim 3, wherein the switch includes a spring that urges the plunger into the nub recess when the nub is not engaged with the plunger.

7. The robotic surgical system of claim 6, wherein the switch includes a footing engaged with the housing, and a button housing coupled to the footing, the button housing movable relative to the footing as the plunger moves relative to the nub recess.

8. The robotic surgical system of claim 7, wherein the spring is engaged with the footing.

9. The robotic surgical system of claim 7, wherein the footing includes a flange that limits movement of the button housing relative to the footing.

10. The robotic surgical system of claim 1, wherein the switch is a dome switch.

11. A surgical instrument for selective connection to a sterile interface module of a robotic surgical system, the surgical instrument comprising:
a housing configured for coupling to an electrosurgical energy source;
an elongated shaft that extends distally from the housing;
an end effector supported on the elongated shaft; and
a switch supported by the housing and positioned to enable electrical energy from the electrosurgical energy source to be transmitted to the end effector when the housing is coupled to the sterile interface module, the switch positioned to prevent the electrical energy from being transmitted from the energy source to the end effector when the housing is uncoupled from the sterile interface module.

12. The surgical instrument of claim 11, wherein the housing defines a nub recess that is configured to engage a nub of the sterile interface module.

13. The surgical instrument of claim 12, wherein the switch includes a plunger that extends into the nub recess and a button disposed within the housing, the plunger positioned to selectively engage the button.

14. The surgical instrument of claim 13, wherein the button is coupled to a printed circuit board.

15. The surgical instrument of claim 14, wherein the printed circuit board flexes toward the button when the plunger engages the button.

16. The surgical instrument of claim 13, wherein the switch includes a spring that urges the plunger into the nub recess.

17. The surgical instrument of claim 16, wherein the switch includes a footing engaged with the housing, and a button housing coupled to the footing, the button housing movable relative to the footing as the plunger moves relative to the nub recess.

18. The surgical instrument of claim 17, wherein the spring is engaged with the footing.

19. The surgical instrument of claim 17, wherein the footing includes a flange that limits movement of the button housing relative to the footing.

20. The surgical instrument of claim 11, wherein the switch is a dome switch.
